(19) United States
(12) Patent Application Publication
(10) Pub. No.: US 2009/0325874 A1
(43) Pub. Date: Dec. 31, 2009

(54) Inhibition of inositol 1,4,5-triphosphate receptor calcium channel/cytochrome c interactions and uses thereof

(76) Inventors: Darren F. Boehning, League City, TX (US); José M. Barral, Galveston, TX (US)

Correspondence Address: Benjamin Aaron Adler;ADLER & ASSOCIATES, 8011 Candle Lane, Houston, TX, 77071, US

(21) Appl. No.: 12/459,253

(22) Filed: Jun. 29, 2009

Related U.S. Application Data
(60) Provisional application No. 61/133,559, filed on Jun. 30, 2008.

(51) Int. Cl. A61K 38/17; A61P 25/28; A61P 35/00

(52) U.S. Cl. 514/12

(57) ABSTRACT

The present invention demonstrates selective pharmacological targeting and inhibition of apoptotic calcium release in lymphocytes from inositol 1,4,5-trisphosphate receptor calcium channels may enhance the immunologic response to tumor cells which have acquired adaptive changes in death receptor signaling to promote survival. The present invention provides methods of protecting lymphocytes from tumor cell-induced apoptotic cell death by contacting lymphocytes with one or more cell permeant peptide(s). Also provided are methods of inhibiting caspase activation and blocking calcium release by blocking binding of cytochrome c to the inositol 1,4,5-triphosphate receptor calcium channel. In addition, methods are provided to treat a cancer, enhance immunologic response by administering or applying small molecule inhibitor, e.g., cell permeant peptides specific for the cytochrome c binding domain of inositol 1,4,5-triphosphate receptor calcium channel.

### CROSS-REFERENCE TO RELATED APPLICATION

This nonprovisional application claims benefit of provisional U.S. Ser. No. 61/133,559, filed Jun. 20, 2008, now abandoned, the entirety of which is hereby incorporated by reference.

### FEDERAL FUNDING LEGEND

This invention was produced using funds from Federal government under National Institutes of Health grant number GM081685. Accordingly, the Federal government has certain rights in this invention.

### BACKGROUND OF THE INVENTION

1. Field of the Invention

The present invention relates to the fields of cell signaling pertaining to lymphocyte cell growth, apoptosis, and cancer cells. More specifically, the present invention discloses a novel apoptotic pathway involving binding of cytochrome c to inositol 1,4,5-triphosphate receptor calcium channel activated in lymphocytes by cancer cells and specific inhibition of this pathway leads to reduced cancer-induced lymphocyte apoptosis.

2. Description of the Related Art

Fas receptor is a member of the tumor necrosis factor, alpha (TNFa) superfamily of death receptors. Fas receptor binds to Fas ligand presented on another cell, initiating a series of events ultimately leading to the activation of pro-apoptotic proteases known as caspases. Fas ligand expression is restricted primarily to activated T cells and natural killer (NK) cells, where it is essential for activation-induced cell death and immune-mediated cytotoxicity. In addition, Fas ligand is constitutively expressed in select immunoprivileged tissues such as the eye and the testis.

Cancer cells can harness the Fas pathway to evade immune attack (1-3). Loss of Fas receptor function is frequently observed in human cancers, reducing the ability of infiltrating T cells and NK cells to kill cancerous cells (4-8). Modulation of Fas signaling for selective survival advantage has been documented in multiple tumor types, including colon, hepatocellular, ovarian and esophageal carcinomas, melanoma, and astrocytoma (1).

Loss of Fas signaling by cancer cells is accomplished at the molecular level by transcriptional down-regulation of Fas receptor or the adaptor protein, Fas-Associated protein with Death Domain (FADD), and up-regulation of negative regulatory components of the Fas pathway such as Fas-Associated protein with Death Domain-like Interleukin-1 beta converting enzyme-inhibitory protein (FLIP), Bcl-XL, and Bcl-2 (2). Moreover, many cancer cells also up-regulate Fas ligand expression, which consequently results in increased T-cell apoptosis and decreased infiltration (9-11). Thus, the identification of the molecular pathways modulating Fas signaling is an important goal in rational drug development that targets this adaptive strategy of cancer cells.

The prior art is deficient in methods of inhibiting inositol 1,4,5-trisphosphate receptor calcium channel-dependent apoptotic signaling. The prior art is also deficient in methods of protecting lymphocytes from apoptotic cell death induced by tumor cells expressing high levels of Fas ligand. The present invention fulfills this long-standing need and desire in the art.

### SUMMARY OF THE INVENTION

The present invention is directed to a method of protecting lymphocytes and other cells from apoptotic cell death induced by tumor cells. Such a method comprises applying cell permeant peptides derived from the cytochrome c binding domain of the inositol 1,4,5-triphosphate receptor calcium channel to the lymphocytes, where the peptide inhibits the release of calcium and thereby inhibits the lymphocyte cell death. Cells to be treated may be in vitro, in vivo or ex vivo. Representative tumor cells include colon cancer cells, cervical cancer cells, lymphoma cancer cells, or other cancer cells.

The present invention is directed to a method of inhibiting cell death due to apoptosis. Such a method comprises regulating calcium release by cytochrome c binding of the inositol 1,4,5-triphosphate receptor calcium channel by contacting the cells with small molecule inhibitors that interfere with the binding of cytochrome c to the inositol 1,4,5-triphosphate receptor calcium channel, thereby inhibiting cell death. Representative diseases which may be treated include cancer, tissue injury caused, for example by burns, stroke, neurodegenerative disease or other diseases or disorders causing apoptosis of cells.

The present invention is also directed to a method to inhibit calcium release by blocking the binding of cytochrome c binding domain to the inositol 1,4,5-triphosphate receptor calcium channel. Such a method comprises interfering with the binding of cytochrome c to the inositol 1,4,5-triphosphate receptor calcium channel thereby inhibiting calcium release. Such inhibition of calcium release may be through use of small molecule inhibitors, or cell permeant peptides.

The present invention is further directed to a method of inhibiting caspase activation. Such a method comprises interfering with calcium release by blocking the binding of cytochrome c to the inositol 1,4,5-triphosphate receptor calcium channel. Such caspase activation inhibition may be used to treat representative diseases such as cancer, cell death, stroke, and neurodegenerative disease.

The present invention is further directed to a method to treating cancer in an individual. Such a method comprises providing a small molecule inhibitor to the individual and the small molecule inhibitor blocks release of calcium mediated by the activation of the inositol 1,4,5-triphosphate receptor calcium channel, and inhibition of apoptosis inhibits cell death of the tumor-specific lymphocytes thus treating the cancer in the individual. Representative cancers which may be treated include lymphoma, colon cancer, or cervical cancer or other cancers.

The present invention is also directed to a methods to enhance the immunologic response against tumors in an individual. Such a method comprises blocking the release of calcium in tumor-specific lymphocytes by applying cell permeant peptides specific for the cytochrome c binding domain of inositol 1,4,5-triphosphate receptor calcium channel thereby inhibiting calcium release and activation of the Fas-dependent death pathway in the lymphocytes thereby enhancing the immunologic response against tumors. Tumors to be treated are colon cancer, cervical cancer, or other cancers.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is noted, however, that the appended drawings illustrate preferred embodiments of the invention and therefore are not to be considered limiting in their scope.

FIGS. 1A-1F show calcium release from inositol 1,4,5-triphosphate receptor calcium channel (IP3R) is required for SW620-mediated killing of Jurkat cells. FIG. 1A shows Fura-2 calcium ratio imaging of SW620/Jurkat co-cultures. The first panel shows only adherent SW620 cells. At a time of 347 s, a single Jurkat cell (indicated by an arrow; second panel), comes into contact with four SW620 cells which leads to a rise in intracellular calcium. FIG. 1B shows the calcium response in the Jurkat cell shown in FIG. 1A. FIG. 1C shows the calcium responses of the four SW620 cells shown in FIG. 1A. Qualitatively similar results were observed in all trials in which a Jurkat cell contacted at least one SW620 cell (data not shown). FIG. 1D shows a histogram of percent Jurkat cells, which released calcium in response to binding SW620 cells after transfection with control RNAi (Bar #1), IP3R-1 RNAi (Bar #2), or co-transfection with IP3R-1 RNAi and the cDNA for the rat IP3R-1 (Bar #3). The total number of transfected cells, which contacted a SW620 cell are indicated. No cells expressing IP3R-1 RNAi (Bar #2) responded (0/15). FIG. 1E shows Caspase-3 like activity (DEVDase activity) in Jurkat cells co-cultured with SW620 cells or cultured alone. Bar #2 shows no pretreatment before co-culture for 24 h with SW620 cells. Bar group #3 show Jurkat cells pre-incubated with the indicated concentrations of aminomethoxy ester form of 1,2-bis(o-aminophenoxy)ethane-N,N,N′,N′-tetraacetic acid (BAPTA-AM) before co-culture. Bar group #4 show Jurkat cells pre-incubated with the indicated concentrations of Xestospongin C (XestC). Bar group #5 show Jurkat cells pre-incubated with the indicated concentrations of B-IP3RCYT. See Methods for details. FIG. 1F shows cell death in Jurkat cells prior to (Control, Bar #1) or 24 h after co-culture with SW620 cells (+SW620, Bar #2). The effects of 1 mM XestC (Bar #3) and 10 nM B-IP3RCYT (Bar #4) were tested by pre-incubation prior to co-culture. The indicated RNAi were transfected 24 h before co-culture as described elsewhere (ref 12) (Bars #5-7). All data represent the mean+/−s.e.m. # indicates p<0.05 relative to control (no incubation with SW620 cells). * indicates p<0.05 relative to co-culture with SW620 cells. ** indicates p<0.05 relative to co-culture with SW620+control RNAi.

FIG. 2 shows calcium and tumor-induced lymphocyte apoptosis. Fas ligand expressed on tumor cells activates Fas receptor on infiltrating lymphocytes. This causes activation of inositol 1,4,5-triphosphate receptor calcium channel by coupling of Fas receptor to phospholipase C-•1 and subsequent production of inositol 1,4,5-triphosphate (IP3) (12). The canonical components of the death-induced signaling complex are also recruited to Fas receptor such as Fas-Associated protein with Death Domain (FADD) and caspase 8/10. Caspase 8/10 activation induces Bid activation and translocation to mitochondria, sensitizing them to calcium-induced cytochrome c release. Cytochrome c subsequently binds to inositol 1,4,5-triphosphate receptor calcium channel, causing further calcium release and mitochondrial calcium overload. Blocking inositol 1,4,5-triphosphate-dependent elevations in cytosolic calcium with Xestospongin C or 1,2-bis(o-aminophenoxy)ethane-N,N,N′,N′-tetraacetic acid (BAPTA) inhibits lymphocyte apoptosis. Specifically blocking cytochrome c binding to inositol 1,4,5-triphosphate receptor calcium channel also inhibits lymphocyte apoptosis, suggesting a viable target for therapeutic intervention.

### DETAILED DESCRIPTION OF THE INVENTION

Intracellular calcium homeostasis is critical to the progression of apoptotic cell death. Inositol 1,4,5-trisphosphate receptor (IP3R) calcium channels regulate intracellular calcium concentration during apoptosis induced by death receptor ligation (12) and cellular damage (13). Apoptosis-specific signaling via inositol 1,4,5-trisphosphate receptor is mediated by cytochrome c, which binds to the calcium channel after being released from mitochondria, resulting in augmented calcium channel function (13). This ultimately leads to cytosolic and mitochondrial calcium overload and cell death. Fas receptor signaling utilizes cytochrome c release from mitochondria to amplify the apoptotic signal (14). Therefore, it was hypothesized that inhibiting inositol 1,4,5-trisphosphate receptor calcium channel-dependent apoptotic signaling in lymphocytes would protect them from apoptotic cell death induced by tumor cells expressing high levels of Fas ligand.

The present invention shows that inhibiting inositol 1,4,5-triphosphate receptor calcium channel function in Jurkat T-lymphoma cells is cytoprotective against apoptosis induced by co-culture with Fas ligand-expressing SW620 colon cancer cells. The inositol 1,4,5-triphosphate receptor calcium channel antagonist Xestospongin C (XestC) and calcium buffering with 1,2-bis(o-aminophenoxy)ethane-N,N,N′,N′-tetraacetic acid (BAPTA) dose-dependently inhibited Jurkat cell apoptosis. Inhibiting exclusively inositol 1,4,5-triphosphate receptor calcium channel activation due to pro-apoptotic signaling in lymphocytes by blocking cytochrome c binding to inositol 1,4,5-triphosphate receptor calcium channel inhibited lymphocyte death with nanomolar affinity. Thus, specifically targeting pro-apopotic signaling through the inositol 1,4,5-triphosphate receptor calcium channel in lymphocytes appears to be a promising therapeutic approach for enhancing tumor cell immunogenicity.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “treating” or the phrase “treating a cancer” includes, but is not limited to, halting the growth of the cancer, killing the cancer, or reducing the size of the cancer. Halting the growth refers to halting any increase in the size, or the number of, or size of the cancer cells, or to halting the division of the neoplasm, or the division of cancer cells. Reducing the size refers to reducing the size of the cancer, or the number of, or size of the cancer cells.

Another chemotherapeutic drug may be administered concurrently or sequentially with the small molecule inhibitors to inhibit release of calcium and subsequent apoptosis used herein. The effect of co-administration with the small molecule inhibitors to inhibit release of calcium and subsequent apoptosis is to lower the dosage of the chemotherapeutic drug normally required and known to have at least a minimal pharmacological or therapeutic effect against a cancer or cancer cell, for example, the dosage required to eliminate a cancer cell. Concomitantly, toxicity of the chemotherapeutic drug to normal cells, tissues, and organs is reduced without reducing, worsening, eliminating, or otherwise interfering with any cytotoxic, cytostatic, apoptotic or other killing or inhibitory therapeutic effect of the drug on the cancer cells.

Small cell permeant peptides encoding the cytochrome c binding domain of the inositol 1,4,5-triphosphate receptor calcium channel can be administered independently, either systemically or locally, by any method standard in the art, for example, subcutaneously, intravenously, parenterally, intraperitoneally, intradermally, intramuscularly, topically, enterally, rectally, nasally, buccally, vaginally, or by inhalation spray, by drug pump, or contained within a transdermal patch, or an implant. Dosage formulations of small cell permeant peptides encoding the cytochrome c binding domain of the inositol 1,4,5-triphosphate receptor calcium channel may comprise conventional non-toxic, physiologically or pharmaceutically acceptable carriers, or vehicles suitable for the method of administration.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Materials

Peptide synthesis of the inositol 1,4,5-triphosphate receptor calcium channel (IP3R) fragment encoding the cytochrome c binding domain of the type I inositol 1,4,5-triphosphate receptor calcium channel isoform and conjugation to BODIPY-577/618 has been described elsewhere (15). This cell-permeant peptide is termed BIP3RCYT. The aminomethoxy ester form of BAPTA (1,2-bis(o-aminophenoxy)ethane-N,N,N′,N′-tetraacetic acid; BAPTA-AM), propidium iodide and fura-2-AM were purchased from Molecular Probes (Eugene, Oreg.). Xestospongin C (XestC) and other chemicals were purchased from Sigma-Aldrich (St. Louis, Mo.).

### EXAMPLE 2

### Cell Culture

Jurkat T-lymphoma (clone E6-1) and SW620 colon adenocarcinoma cells were purchased and cultured according to the guidelines of American Type Culture Collection (ATCC, Manassas, Va.). SW620 cells grow as adherent monolayers, whereas Jurkat cells grow in suspension. Jurkat cells were co-cultured with SW620 cells by exchanging the culture medium of SW620 cells (80 percent confluent in a 10 cm dish) with 10 ml Jurkat medium containing 1×10⁶ Jurkat cells. Co-cultures were incubated for 24 h, after which cell death or caspase activity was determined in Jurkat cells. Pre-loading 1,2-bis(o-aminophenoxy)ethane-N,N,N′,N′-tetraacetic acid (BAPTA) or B-IP3RCYT into Jurkat cells was performed for 30 min at room temperature. Free BAPTA-AM or B-IP3RCYT was removed by media exchange prior to addition to SW620 cells. XestC was similarly pre-incubated with Jurkat cells prior to co-culturing, and remained in the culture medium for the duration of the co-culture experiment.

### EXAMPLE 3

### RNAi

Knockdown of the human inositol 1,4,5-triphosphater receptor-1 (IP3R-1) in Jurkat cells and rescue with the rat IP3R-1 isoform was done as described elsewhere (12). The rat IP3R-1 has several base substitutions within the targeted region of the human IP3R-1 gene allowing expression in the presence of this RNAi. Control experiments utilized an RNAi with several deletions and insertions (12). Co-transfection with yellow fluorescent protein (YFP) was utilized to identify cells containing the RNAi for calcium imaging and cell death assays (see in Example 4).

### EXAMPLE 4

### Calcium Imaging

SW620 cells grown on cover slips were loaded with fura-2-AM for 30 min at room temperature. Jurkat cells were loaded in suspension as described (12). Cover slips with fura-2 loaded SW620 cells were placed in an imaging chamber (ATTOFLUOR® cell chamber, Molecular Probes) and imaged at 40× magnification as described (12). Fura-2 loaded Jurkat cells (˜1×10⁴) were added by solution exchange. In approximately 20% of trials (6 of 28), a Jurkat cell came into direct contact with one or more SW620 cells in the field of view. To measure responses in Jurkat cells co-transfected with RNAi and yellow fluorescent protein (YFP), images were acquired with a 20× objective and ˜5×10⁴ Jurkat cells were added to the imaging chamber containing SW620 cells. This methodology permitted imaging of the low abundance transfected Jurkat cells contacting SW620 cells with greater frequency at the expense of resolution. RNAi data is expressed as a histogram of the percentage responding YFP positive cells from a minimum of 15 cells, which were confirmed to directly contact an SW620 cell. A response was defined as an increased in 340/380 ratio greater than 0.5.

### EXAMPLE 5

### Caspase-3 Activity and Cell Death Determination

Caspase activity and cell death were determined specifically in Jurkat cells by removing the medium containing the Jurkat cells into a fresh tube. An additional rinse with culture medium was used to fully recover the Jurkat cells. Adherent SW620 cells remained attached to the culture plate after this rinse as determined by light microscopy. Jurkat cells were pelleted by centrifugation at 1000 g and cell death was determined by propidium iodide staining as described (12). Fluorometric determination of DEVDase (caspase-3 like) activity was performed on lysates prepared from Jurkat cells as described (12, 15).

### EXAMPLE 6

### Statistical Analysis

Statistical significance was determined by using unpaired two tailed t tests on data points from at least three separate experiments. Data comparisons were considered significant if p was <0.05.

### EXAMPLE 7

### Results and Discussion

SW620 colon carcinoma cells express high levels of Fas ligand, and have been shown to be potent inducers of Jurkat apoptosis in a Fas dependent manner (16). Thus, co-culture of Jurkat cells with SW620 cells should induce pro-apoptotic calcium release in Jurkat cells, and that this calcium release should be required for lymphocyte apoptosis. To monitor intracellular calcium in co-culture experiments, SW620 cells were grown on cover slips and loaded with fura-2. Jurkat cells were also separately loaded with fura-2 to simultaneously image intracellular calcium in both cell types. After establishing baseline calcium levels in SW620 cells, Jurkat cells were added to the imaging chamber by solution exchange. In 6 out of 28 trials, a Jurkat cell bound directly to one or more SW620 cells.

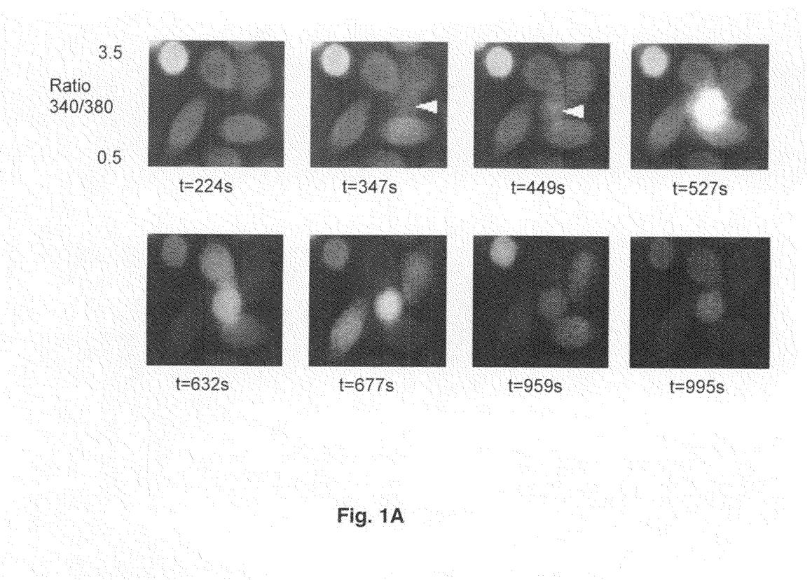

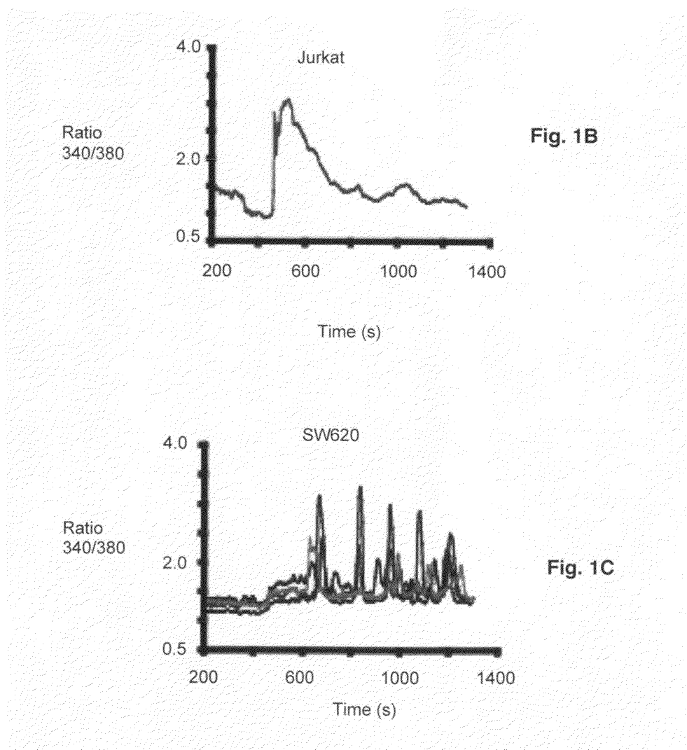

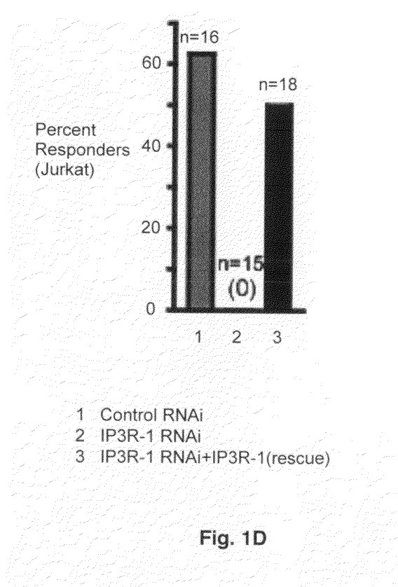

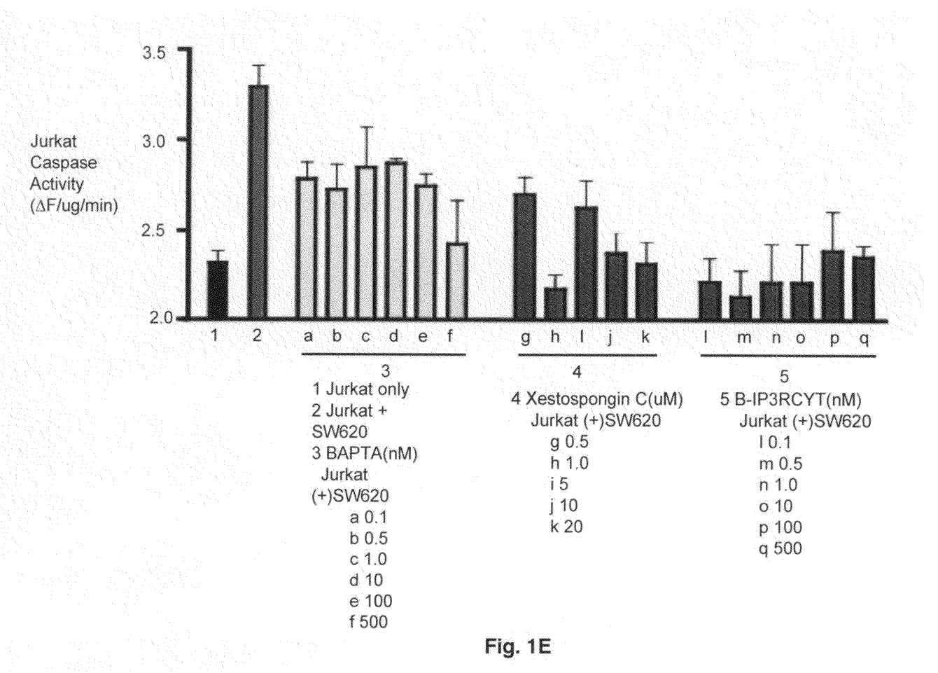

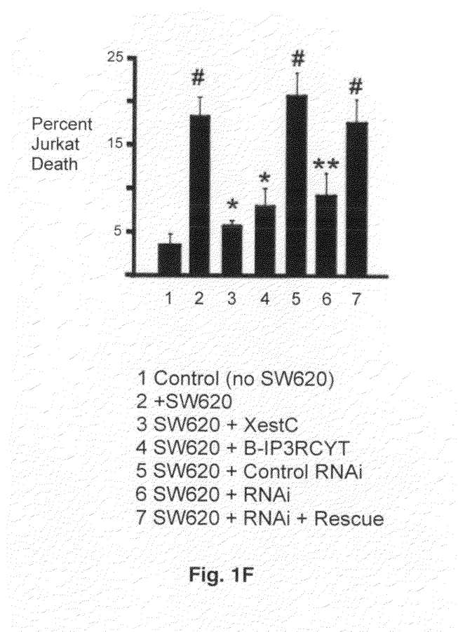

As shown in FIGS. 1A-1B, upon binding SW620 cells, a large transient rise in intracellular calcium is induced in Jurkat cells. Interestingly, after a short delay, calcium oscillations are induced in SW620 cells (FIGS. 1A, 1C). This finding is of significance, since coupling of Fas ligand to calcium release in Fas ligand presenting cells has not been demonstrated. Fas ligand microvesicles can induce calcium release in Jurkat cells via inositol 1,4,5-triphosphate receptor calcium channel (12, 15). To determine if the calcium response in Jurkat cells was due to inositol 1,4,5-triphosphate receptor calcium channel-mediated calcium release, inositol 1,4,5-triphosphate receptor calcium channel levels were knocked down with a previously characterized double stranded RNAi oligo targeting the human inositol 1,4,5-triphosphate receptor-1 (IP3R-1) isoform (12).

Knockdown of IP3R-1 completely abrogated Jurkat calcium release in response to SW620 cell binding (0/15 cells; FIG. 1D). Importantly, calcium release could be rescued by co-transfecting the rat IP3R-1 gene (9/18 cells). As expected, Jurkat cells expressing control RNAi retained the ability to release calcium after contact with SW620 cells (10/16 cells). These results indicate that binding to SW620 cells induces IP3R-dependent calcium release in Jurkat cells.

To examine the effects of modulators of inositol 1,4,5-triphosphate receptor calcium channel-dependent calcium release on SW620-dependent Jurkat apoptosis, caspase-3 activation was monitored. Co-culture of Jurkat cells with SW620 cells induced significant activation of caspase-3 in Jurkat cells (FIG. 1E; # p<0.05 relative to Jurkat cells which were not co-cultured with SW620 cells).

To determine if intracellular calcium contributed to this increase in caspase-3 activity, Jurkat cells were loaded with various concentrations of the calcium chelator BAPTA-AM to buffer cytosolic calcium levels. Significant attenuation of caspase-3 activation was observed by incubating the cells with 100 and 500 nM BAPTA-AM (*p<0.05 relative to Jurkat cells co-cultured with SW620 cells without pre-treatment).

To determine if the inositol 1,4,5-triphosphate receptor calcium channels are required for lymphocyte apoptosis, cells were incubated with various doses of the cell permeant inositol 1,4,5-triphosphate receptor calcium channel inhibitor Xestospongin C (XestC), a selective blocker of inositol 1,4,5-triphosphate receptor calcium channel function when used at low micromolar concentrations (17, 18). XestC inhibited caspase-3 activation in Jurkat cells at concentrations ranging from 1 to 20 micromolar, indicating that inositol 1,4,5-triphosphate receptor calcium channel activity was required for caspase-3 activation (FIG. 1E). Importantly, XestC potently blocked Jurkat cell death induced by co-culture with SW620 cells (FIG. 1F).

During apoptosis, calcium release from the inositol 1,4,5-triphosphate receptor calcium channel is augmented by direct binding of cytochrome c to the channel (12, 13, 15). Cytochrome c binds to the cytosolic C-terminal “tail” of the inositol 1,4,5-triphosphate receptor, decreasing the ability of calcium to inhibit the channel and prolonging calcium release (15). Peptides derived from the cytochrome c binding domain of inositol 1,4,5-triphosphate receptor, when introduced into cells, inhibit calcium-dependent apoptosis without altering agonist-induced calcium release (12, 13, 15). Coupling of one of these peptides to the hydrophobic dye BODIPY 577/618 renders the peptide as cell permeable, while retaining anti-apoptotic activity (12, 15). Thus, whether this peptide, termed B-IP3RCYT, could block SW620-dependent cell death of Jurkat cells was tested.

As shown in FIG. 1E, B-IP3RCYT inhibited caspase-3 activation in Jurkat cells at nanomolar concentrations. This result is consistent with the in vitro observation that IP3RCYT has a higher affinity for cytochrome c than the cytosolic tail of inositol 1,4,5-triphosphate receptor calcium channel, and can inhibit Fas-dependent cell death induced by Fas ligand microvesicles or the cytotoxic drug staurosporine at nanomolar concentrations (15). Importantly, the B-IP3RCYT peptide also inhibited Jurkat cell death induced by SW620 co-culture (FIG. 1F). Thus, specifically blocking apoptotic calcium release via the inositol 1,4,5-triphosphate receptor calcium channel inhibits lymphocyte apoptosis induced by Fas ligand-expressing cancer cells. Finally, RNAi of IP3R-1 also inhibited Jurkat cell death, and these effects were reversed by rescue with the rat IP3R-1 gene (FIG. 1F).

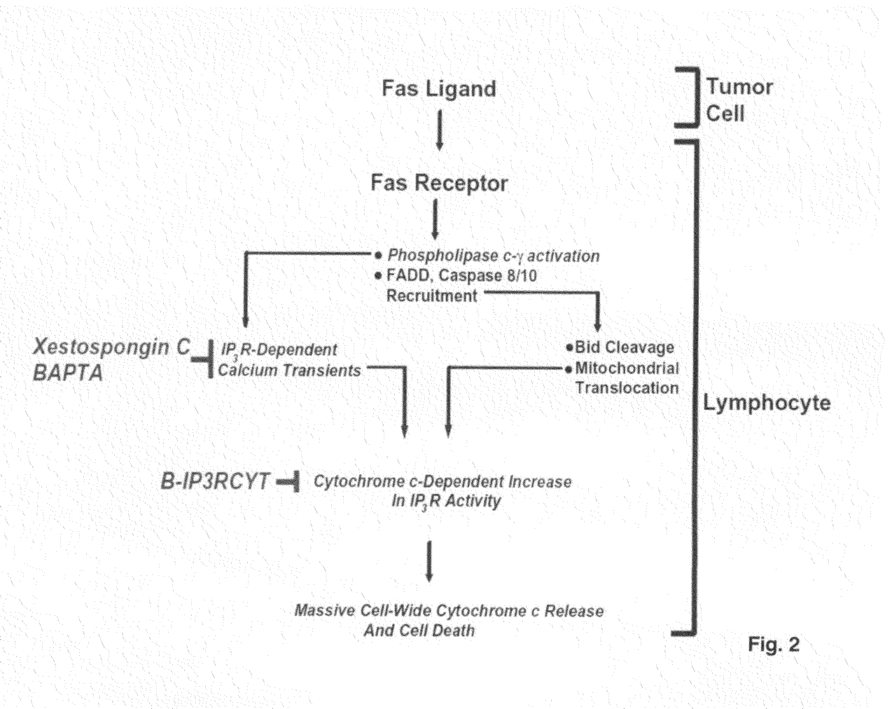

The ability of tumor cells to attain immunoprivileged status in vivo by up-regulating Fas ligand has remained relatively controversial (9, 11, 19). In the present invention, it was demonstrated that in vitro Fas-dependent killing of a lymphocyte cell line by cancer cells requires calcium release from inositol 1,4,5-triphosphate receptor calcium channel. Identification of the inositol 1,4,5-triphosphate receptor calcium channel as a mediator of lymphocyte apoptosis induced by tumor cells highlights a novel Fas-dependent pathway, which warrants further investigation in vivo (see FIG. 2). Significantly, lymphocyte apoptosis could be inhibited by specifically blocking apoptotic calcium release from the inositol 1,4,5-triphosphate receptor calcium channel. Lymphocyte apoptosis was inhibited by B-IP3RCYT when placed in the culture medium at low nanomolar concentrations. Since B-IP3RCYT exhibits no significant cellular toxicity and does not modify agonist-induced calcium release (12, 15), targeting I inositol 1,4,5-triphosphate receptor calcium channel/cytochrome c interactions appears to be an attractive target for enhancing the immunologic response to tumors, which have acquired adaptive changes in death receptor signaling to promote survival.

The following references were cited herein:
1. Reichmann E 2002, Semin Cancer Biol 12(4), 309-315.
2. Houston A, and O'Connell, J, 2004, Curr Opin Pharmacol 4(4), 321-326.
3. Ryan, A E, et al, 2006, Cell Cycle % (3), 246-249.
4. O'Connelm J, et al, 2001, Nat Med, 7(3), 271-274.
5. Houston, A, et al; 2003, Int J Cancer, 107(2), 209-214.
6. Bebenek, M et al, 2006, Med Sci Monit, 12(11), CR457-461.
7. Yamana K, et al, 2005, Br J Cancer, 93(5), 544-551.
8. Reimer T, et al, 2002, Breast Cancer Res 4(5), R9.
9. Ryan, A E, et al, 2005, Cancer Res 65(21), 9817-9823.
10. Bennett, M W, et al, 2001, J Clin Pathol 54(8), 598-604.
11. Houston, A, et al, 2003, Br J Cancer, 89(7), 1345-1351.
12. Wozniak A L, et al, 2006, J Cell Biol 175(5), 709-714.
13. Boehning D, et al, 2003, Nat Cell Biol, 5(12), 1051-1061.
14. Scaffidi, C, et al, 1998, Embo J, 17(6), 1675-1687.
15. Boehning, D, et al, 2005, Proc Natl Acad Sci USA 102:1466-71.
16. O'Connell J, et al, 1996, J Exp Med, 184(3), 1075-1082.
17. Gafni J, et al, 1997, Neuron, 19(3), 723-733.
18. Ta T, et al, 2006, Mol Pharmacol, 69(2), 532-538.
19. Igney and Krammer, 2005, Cancer Immunol Immunother 54(11), 1127-1136.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of protecting lymphocytes from apoptotic cell death induced by tumor cells, comprising:
contacting one or more cell permeant peptide(s) to said lymphocytes, whereby said peptide(s) inhibit apoptotic cell death induced by tumor cells.

2. The method of claim 1, wherein said cell permeant peptide(s) inhibit the release of calcium from inositol 1,4,5-triphosphate receptor calcium channels in said lymphocyte, and said inhibition of calcium protects said lymphocyte from apoptotic cell death.

3. The method of claim 1, wherein said apoptotic cell death in said lymphocytes is induced by said tumor cells by binding of the Fas receptor on said lymphocytes to the Fas ligand on said tumor cells, whereby said binding activates said apoptotic cell death in said lymphocytes.

4. The method of claim 1, wherein said application of cell permeant peptide(s) to said lymphocytes is in vitro, in vivo, or ex vivo.

5. The method of claim 1, wherein said cell permeant peptides are derived from the cytochrome c binding domain of the inositol 1,4,5-triphosphate receptor calcium channel.

6. The method of claim 1, wherein said tumor cells are colon cancer cells, cervical cancer cells, or other cancer cells.

7. A method of inhibiting apoptosis in lymphocytes, comprising:
regulating calcium release in said lymphocytes activated by Fas receptor-Fas ligand binding on said lymphocyte, whereby said regulation of calcium release inhibit apoptosis in said lymphocytes.

8. The method of claim 7, whereby said calcium release is regulated by cytochrome c binding to the inositol 1,4,5-triphosphate receptor calcium channel.

9. The method of claim 8, wherein said calcium release is regulated by one or more small molecule inhibitor(s) that interfere with said binding of cytochrome c to the inositol 1,4,5-triphosphate receptor calcium channel, thereby regulating said calcium release.

10. The method of claim 7, whereby said inhibiting of apoptosis in lymphocytes is used to treat cancer, stroke, neurodegenerative disease, or other diseases or disorders.

11. The method of claim 10, whereby said cancer is colon cancer, cervical cancer, or other cancers.

12. The method of claim 10, whereby said neurodegenerative disease is Parkinson's disease, Alzheimer's disease, amyotrophic lateral sclerosis disease, dementia, or other neurodegenerative diseases.

13. A method to inhibit calcium release in lymphocytes comprising:
blocking the binding of cytochrome c to inositol 1,4,5-triphosphate receptor calcium channel, wherein said blocking inhibits calcium release.

14. The method of claim 13, wherein said blocking of binding of cytochrome c to inositol 1,4,5-triphosphate receptor calcium channel is by application of small molecule inhibitor(s).

15. The method of claim 14, wherein said small molecule inhibitor, is one or more cell permeant peptide(s) or other small molecule inhibitors.

16. A method to inhibit caspase activation in a cell, comprising:
interfering with calcium release in said cell by blocking binding of cytochrome c to the inositol 1,4,5-triphosphate receptor calcium channel, whereby said blocking inhibits caspase activation in the cell.

17. The method of claim 16, wherein said caspase activation inhibition is used to treat disease or disorders including cancer, apoptosis, stroke, or neurodegenerative disease.

18. The method of claim 17, wherein said cancer is colon cancer, cervical cancer, lymphoma, or other cancers.

19. The method of claim 16, wherein said interfering with calcium release uses small molecule inhibitors which include cell permeant peptides derived from the cytochrome c binding domain of the inositol 1,4,5-triphosphate receptor calcium channel or other small molecule inhibitors.

20. A method to treat cancer in an individual, comprising:
administering a small molecule inhibitor to the individual, wherein said small molecule inhibitor blocks release of calcium mediated by the activation of the inositol 1,4,5-triphosphate receptor calcium channel, thereby inhibiting cell death of the tumor-specific lymphocytes thereby treating said cancer.

21. The method of claim 20, wherein said cancer is colon cancer, or cervical cancer, or other cancers.

22. The method of claim 20, wherein said small molecule inhibitor is a cell permeant peptide or other small molecule inhibitor.

23. A method to enhance immunologic responses to tumors in an individual, comprising:
applying cell permeant peptides specific for the cytochrome c binding domain of inositol 1,4,5-triphosphate receptor calcium channel to block calcium release thereby inhibiting calcium release and apoptotic cell death in lymphocytes, thereby enhancing the immunologic response to said tumors in the individual.

24. The method of claim 23, wherein said tumors are colon cancer, cervical cancer or other cancers.
